(19) United States
(12) Patent Application Publication
(10) Pub. No.: US 2004/0057917 A1
(43) Pub. Date: Mar. 25, 2004

(54) Pharmaceutical composition for treatment and /or prevention of a light-dermatosis

(75) Inventors: Peter Wolf, Graz (AT); Karl-Heinz Dolinschek, Graz (AT)

Correspondence Address: Ursula B. Day, Suite 3220, 350 Fifth Avenue, New York, NY, 10118, US

(73) Assignee: HANS-DIETER PRENTNER, Gratwein (AT)

(21) Appl. No.: 10/431,624

(22) Filed: May 7, 2003

(30) Foreign Application Priority Data
Sep. 24, 2002 (DE) 202 14 753.3

(51) Int. Cl. A61K 7/42; A61K 35/78

(52) U.S. Cl. 424/59; 424/767

(57) ABSTRACT

A pharmaceutical composition for treatment and prevention of light dermatosis includes (i) a medical drug form nopal cactus, and (ii) at least one active substance with light-dermatosis therapeutic properties and wherein the pharmaceutical composition is provided in a suitable form for topical application.

### CROSS-REFERENCES TO RELATED APPLICATIONS

This application claims the priority of German Patent Application, Serial No. 20214 753.3, filed Sep. 24, 2002 pursuant to 35 U.S.C. 119(a)-(d), the disclosure of which is incorporated herein by reference.

### BACKGROUND OF THE INVENTION

The present invention relates to a pharmaceutical composition comprising (i) a medical drug from nopal cactus and (ii) at least one active substance having light-dermatosis-therapeutic properties for the treatment and/or prevention of a light-dermatosis.

Furthermore, the present invention refers to a method for producing a pharmaceutical composition comprising (i) a medical drug from Nopal cactus and (ii) at least one active substance having light-dermatosis-therapeutic properties for the treatment and/or prevention of light-dermatoses, in particular light dermatitis solaris (sun burn), and its use in topically effective medical drug formulas. In addition, the present invention refers to a pharmaceutical composition, suitable for use in skin tanning and/or for enhancing the formation of pigmentation in the skin.

Skin cancer is a serious human health problem. Incidences of non-melanoma-skin cancer (basal cell carcinoma and squamous cell carcinoma) in the United States of America in the 90s was annually 900,000 and 50,000 for melanomas. In the 90s, the annual death rate was about 2,000 for non-melanoma skin cancer, respectively 6,000 for melanomas and if the current trend continues in the next eighty years, 800,000 deaths are expected due to skin cancer.

The causal relationship between non-melanoma skin cancer and the chronic radiation effect of ultraviolet light of the sun was clearly shown whereby the effect of the sun is also an important trigger factor for melanoma. It is generally known that one of the main targets for ultraviolet light damage leading to cancer—is the DNA.

Whereas in former times, tanned skin was a sign of physical country labor, and light, untanned skin was desirable as a status symbol, nowadays, tanned skin signals activity, health, sportyness and success. In order to realize a fast and deep tanning when sun bathing, many expose themselves knowingly to the sun light without being aware of the acute and chronic effects of it.

The radiation spectra of the sun are very broad and only the narrow range of wavelengths from 400 to 800 nm is visible for the human eye. In the direction of the larger wavelengths follows the infrared range (IR) which is detected as warmth. In the short-wave range the visible light follows the ultraviolet range. Due to their different physiological effect, different UV ranges have been defined.

The UV-C-range comprises the wavelengths from 100 to 280 nm. This wavelength range is entirely absorbed through the ozone layer in the stratosphere of the earth and is therefore not contained in the natural spectrum of sunlight as measured, for example, at sea level on the earth's surface. The UV range comprises the wavelengths from 280 to 315 nm and causes sunburn as well as indirect skin tanning. The UV-A-range comprising the wavelengths of 315 to 400 nm rarely leads to sunburn; however it effects direct skin tanning.

The sunlight has benevolent as well as also damaging effect on the skin and the organism. At low doses, sun radiation raises the well being of the body as well as the body's capacity. The UV-B portion of the sunlight promotes the vitamin D synthesis and the formation of the skin's own self-protection. This self-protection includes predominantly pigmentation (tanning of the skin) and in the thickening of the horny layer of the skin (formation of a heloma). At the same time, excessive radiation to the skin with UV-B triggers acute damage such as formation of a sunburn which, depending on the doses of UV and insensitivity of the skin, ranges from a reddening up to a strong burn with blister formation. Chronic UV-B damage, for example damage after continuous chronic UV-B impact for years, are premature aging of the skin in form of structural changes in the tissue of the skin, in the connective tissue of the skin and, in extreme cases, to a neoplastic change (among others, also after repeated impact of extreme high UV single dosages) the formation of skin cancer.

UV-A radiation causes the direct pigmentation and amplifies the biological effect of UV-B radiation positively as well as negatively. Since the UV-A radiation is a long-wave radiation, which can penetrate into the connective tissue of the skin, premature or aging of the skin is prevalent, particularly through simultaneous UV-B radiation. The direct pigmentation triggered through the UV-A portion of the sunlight is generally relatively low and does not facilitate tanning without indirect pigmentation.

It is known today that intensive UV-A as well as also UV-B radiation damages the DNA in the cell nucleus of skin cells through photochemical secondary reactions. Thereby, free radicals react with the DNA components and alter these.

The human organism possesses a multitude of repair mechanisms. On the one hand, the cell cycle can be stopped until the damage in the DNA components are repaired. On the other hand, the transmission of faulty information to the daughter cells can be prevented, whereby cells with irreparable DNA damage are programmed for cell death (apoptosis). By eliminating these cells, formation of degenerate cells, which turn into malignant tumors, can be prevented.

It is known today that high and repetitive radiation impact overtaxes the body's own natural repair mechanisms and thereby exhaust the so-called “sun capital” so that with passage of time, acute and chronic light-dermatosis can occur, in particular through UV damage, which is only conditionally reversible as well as chronic light damage up to skin cancer. This weakens not only the cells themselves but weakens respectively, damages also the immune system, which can prevent cancer growth.

It would therefore be desirable and advantageous to provide an improved preventive measures which obviates prior art shortcomings and which eliminates the afore-described problems

### SUMMARY OF THE INVENTION

According to one aspect of the present invention a pharmaceutical composition is provided having light-dermatosis-therapeutic properties and strengthens the pigment formation in the skin. Through the strengthened pigment formation on the one hand, the natural sun protective effect of the skin is raised, and on the other hand, a deeper skin tanning is realized, which is further defined in the following paragraph.

In accordance with another aspect of the present invention, a method is provided for producing this pharmaceutical composition used in the treatment and/or prevention of a light-dermatosis.

The present invention resolves prior art problems by providing a pharmaceutical composition comprising (i) a medical drug from and cactus and (ii) at least an effective and active substance with light-dermatosis therapeutic properties for treatment and/or prevention of light-dermatosis. At the same time, the pigment formation in the skin and thus, tanning of the skin, is not inhibited but amplified.

The present invention refers thus to a pharmaceutical composition comprising (i) a medical drug from nopal cactus and (ii) at least one active substance with light-dermatosis therapeutic properties.

A “medical drug” is defined as a fresh or dried medical plant or its parts, such as for example, roots, bark, leaves, blossoms, seeds, fruits and secretions, for example of essential oils (see Hunnius; Pharmaceutical Dictionary, 8. Edition, de Gruyter, 1998; ISBN 3-11-015793-4). The medical drug from nopal cactus comprises a pharmaceutical effective compound of a nopal cactus.

The Nopal cactus is one of the more than hundred fig cactus species types known in Mexico. Subspecies of the Nopal cactus are in particular Opuntia ficus Indica and Opuntia ficus streptacanthia Lemaire. The term “Nopal cactus” is defined herein as all fig cactus species types, in particular Opuntia ficus streptacanthia Lemaire, preferably Opuntia ficus Indica.

The Nopal plant called fig cactus in German, carries the genus name Opuntie belonging in the taxonomic classification to the Cactaceaes. In Spanish, the fig cactus is also called nopal or also nopal carcon. Likewise, commonly used are also the names nopalito in Mexico, tuna, which is otherwise the name for the fruit, in Argentina and Chile also known as nopalnocheztli. All of these names are comprised in the foregoing under the term Nopal cactus.

In Mexico, the nopal cactus is used traditionally as a vegetable and also as a natural medication. It is known that nopal is rich in iron, calcium, potassium, magnesium, manganese, silicon, aluminum and furthermore in amino acids, vitamins A, B1, B2, B3 and C, but also in resins, in tannins and carotene. In addition, the nopal cactus has an extraordinary high portion of pectin. It is long known that nopal cactus can lower an elevated cholesterol level, respectively triglyceride level, can lower blood sugar levels that are too high in patients with diabetes type 1 and type 2, as well as inhibit the uptake of glucose in the intestine and aids in reducing weight (The Healing Forces of Nopal Cactus; H. Bankhofer, K. -H. Dolinchek, F. Rheinisch, Kneipp Publishers, 1. Edition 2002 ISBN 3-902191-01-5, p. 7 to 13).

Surprisingly, it has been found that a medical drug from nopal cactus, in particular from nopal cactus and DNA repair enzymes, exhibit light-dermatosis-therapeutic properties, in particular anti-inflammatory and skin soothing properties defined as follows. In addition, the medical drug from nopal cactus, in particular from nopal cactus in combination with DNA—repair enzymes does not effect an inhibition of pigment formation due to UV radiation in the skin, but supports an amplification of pigment formation due to UV radiation and thus an amplified tanning of the skin.

The medical drug from nopal cactus can comprise roots, leaves (branches), blossoms, seeds, fruits and cactus secretion (muzilago), in particular from cactus branches and cactus secretion. Particularly preferred, the medical drug consists of cactus secretion (muzilago) which is particularly contained in the cactus branches (pencas). The term “branches” (pencas) is defined herein as a botanical term oftentimes erroneously defined as leaves.

In a preferred embodiment, the medical drug from nopal cactus is produced through comminution of thornless cactus branches. Especially preferred are the branches (pencas) of the nopal, in particular Opuntia ficus Indica, at an age of one to two years. For this, the nopal branches are preferably washed by machine, dried and thereafter the spikes burned of with gas flames. After that, they are thoroughly washed a second time. The nopal are then hacked, chopped and thereafter slowly dried in drying installation over the course of several days respectively weeks at about 30° C. The dried nopal pieces are ground to a fine powder and centrifuged to separate the heavy components (fibers) to thereby eliminate them. Especially important for the production is the finely ground powder with the muzilago component (cactus secretion). In an especially preferred embodiment, the nopal powder so obtained, subsequently will be sifted through a screening system in order to remove larger particles. In a further treatment step, it is an object to obtain the finest possible powder. The powder is sterilized by means of UV radiation.

Alternatively, the commercially available nopal powder (obtained from the Hando company, Graz, Austria) can also be used as medical drug. Dependent on need, the nopal powder can additionally be subjected to and/or purified by further treatment steps, in particular through centrifugation, in order to remove fiber particles and larger particles from the powder and in order to realize an improved formulation of the end product.

The term “light-dermatosis” within the scope of the present patent application is an acute and/or chronic light-dermatosis defined as follows:

Light-dermatosis, or also called photo dermatosis may be an alteration and/or damage of the skin due to impact of light, in particular ultraviolet radiation. Light-dermatosis can be the physiological reactions of the skin as for example a sunburn, increased formation of melanin (hyper pigmentation), akanthosis and hyperkeratose (thickening of the skin due to light impact) as well as pathological reactions of the skin, such as for example diseases from the family of the so-called sun allergies (hyper sensitivity reactions to sunlight), premature aging of the skin and/or skin cancer.

Sun allergies can be photo dermatosis in a medical sense, such as for example the polymorphous light-dermatosis, lupus erythematosis or drug induced light reactions.

Sun dermatoses can occur at normal or abnormal functional situation in the naturally occurring cellular repair mechanisms. In particular, in diseases (such as for example, xeroderma pigmentosum) characterized by abnormal repair capacity for UV induced damage in the desoxyribonucleic acid (DNA), which codes for the body's own genetic substance, acute and/or chronic photo dermatoses tend to occur already in childhood.

An acute light-dermatosis, among others, may be, in particular, dermatitis solaris (sunburn) which is characterized by a photo traumatic reaction at normal light sensitivity through an overdose of UV light and which comprises, at the cellular level, an acute DNA damage, and clinically, exhibits the symptoms of reddened skin, inflamed skin, eventually blister formation and at a later stage, flaking of the light exposed skin area. In addition, an acute light-dermatosis can also be a sun allergy (hypersensitivity reactions to sunlight) in particular, a polymorphous light-dermatosis, lupus erythematodes or a drug induced light reaction.

The chronic light-dermatosis may be predominantly based on DNA damage and/or consequences therefrom and in particular, tissue degeneration, premature aging of the skin, benign and malignant cell changes, in particular, atrophy of the epidermis and degeneration of the connective tissue in the dermis through repeated overexposure by the sun accompanied by a coarsening of the skin profile, cysts, comedons, keratoses and/or the more frequently appearing squamous cell carcinoma and malignant melanoma (see Pschyrembel, Clinical Dictionary, 257. Edition, de Gruyter, 1994, ISBN 3-11-01692-3). The consequences of an acute and/or chronic light-dermatosis can also occur in inner organs, as for example, in systemic photoallergies and/or lupus erythematodes.

As used herein, “therapy” is defined herein as curing, alleviating or prevention (prophylactic) of abnormal conditions or diseases.

The term “light-dermatosis therapeutic properties” is defined as anti-inflammatory (antiphlogistic), skin soothing and/or DNA damage repairing property. The expert has at his disposal all those substances with an active substance that is anti-inflammatory, skin soothing, DNA-damage repairing property. In a particularly preferred embodiment, the active substance, respectively the active substance having anti-inflammatory and skin soothing, for example, synthetically produced and/or nature-identical active substances and/or extracts or and/or active ingredients from medicinal plants, for example, chamomile, aloe vera, witch hazel, etc. A preferred active substance is bisabolol, panthenol, dexpanthenol, allantoin, vitamin E, extract form green tea, chamomile, aloe vera, witch hazel, in particular panthenol, dexpanthenol, allantoin, reishi (ganoderma lucidum), extract from green tea, chamomile and/or vitamin E.

An active substance having DNA-damage repairing properties may be at least one DNA repair enzyme, in particular endonuclease V, O⁶-methylguanine-DNA-methyltransferase, photolyase, uracil-and hypoxanthin-DNA-glucosylase, apyrimidin, apurin-endonuclease, DNA exonuclease, damaged base glycosylase, in particular 3-methyladenine-DNA-glycosylase and/or Correndonuclease, or the DNA repair system is contained in micrococcus luteus-extract and/or anacystis nidulans-extract.

In a special embodiment, at least one DNA-repair enzyme may be contained in a carrier material, wherein the carrier material is selected from liposomes, nanocapsules, nanoparticles and microparticles, in particular, liposomes. The liposomes can be unilamellar and/or in particular multilamellar liposomes. The production and use of the carrier material are known to those skilled in the arts and are also described, for example, in Rudolph Voigt; Pharmaceutical Technology 9th edition, German Pharmacists Press, 2000, Chapter 25.

It a particularly preferred embodiment, the pharmaceutical composition can comprise the medical drug from the nopal cactus in combination with endonuclease V, photolyase and/or micrococcus luteus and/or anacystis nidulans-extract in liposomal form.

Alternatively, an extract from micrococcus luteus (yellow micrococcus) which contains UV-endonuclease with the DNA glycosylase AP (apyridin/apurin) containing endonuclease activity, in multilamellar liposomal form can be utilized, in particular, commercially available as “ultrasome” (AGI Dermatics Inc. Freeport, N.Y. USA). In addition, an extract which is obtained from plankton anacystis nidulans likewise can be used, containing photolyase in multilamellar, liposomal from in particular in the commercially available “photosome” (AGI Dermatics Freeport, N.Y., USA).

In a further preferred embodiment, the pharmaceutical composition can contain about 124 parts by weight, preferably about 2 to 25 parts by weight, in particular about 2 to 15 parts by weight of the compound (i) and about 1 to 40 parts by weight, preferably about 2 to 25 parts by weight, particularly preferred about 2 to 15 parts by weight of the compound (ii), and as a residual portion (v), one or more additional additives, with respect to 100 parts by weight of a composition with the compounds (i), (ii) and (v).

Additives, within the scope of the present invention are emulsifiers, lipids, substances to adjust the pH value, preservatives, dyestuffs, perfume—and aroma substances, supplemental and carrier material, light protection substances, active substances against insects, skin tanning products, tanning accelerators, vitamins, active substances and/or complexes of active ingredients, as for example moisturizer and/or anti-aging complexes and/or supplements for formulation.

As emulsifiers, anionic, ionic, or non-ionic (neutral) tensides can be used; for example alkaline soaps, metal soaps, amino soaps, sulfurized and sulfonized compounds, invert soaps, high alcohols of fatty acids, higher alcohols, in particular, fatty alcoholethoxylate, in particular glycerin, partial fatty acid of sorbitant and polyoxyethylene sorbitans, i.e. lannette types, wool wax, lanolin, or other synthetic products for producing of oil/water-and/or water/oil emulsions.

Lipids as used can be in the form of fatty and/or oil-type and/or wax-type components for production of salves, cream, lotions or emulsions and can be Vaseline, silicone, natural or synthetic waxes and fats, fatty acids, fat alcohols, fatty acid esters, for example, mono-, di-, or triglycerides, paraffin oil or vegetable oil, hardened caster oil or coconut oil, pork fat, synthetic fats, such as for example capryl-, caprin, laurin- and stearin acid-based, such as for example Softisan® or triglycerides mixtures as well as Miglyol®.

For adjusting the pH-value, osmotically effective acids and bases can be used for example, such as hydrochloric acid, citric acid, sodium hydroxide, potassium hydroxide sodium hydrogencarbonate, organic acids and buffering systems, such as for example citrate, phosphate, tris buffer or triethanolamine. In a preferred embodiment, organic acids, hydroxicarbon acids, such as for example fruit acids, preferably milk acid, phenyl glycollic acid, malic acid, tartaric acid and racemic acid.

To increase stability of the composition, conservation agents can be added, such as for example glycerin, phenoxyethanol, methyl- or propylbenzoate (parabene) methyl dibromoglutanitril, hydantoin, jodpropinylbutylcarbamat, quarternary compounds, triclosan, sorbic acid and/or their salts, in particular potassium sorbate and benzoic acid and/or their salts, in particular, sodium benzoate, preferably glycerin, potassium sorbate and sodium benzoate.

In addition, dyestuffs can be added, preferably all dyestuffs certified for food and/or cosmetics respectively, those that are known to those skilled in the arts, for example, yellow and/or red iron oxide and/or titanium oxide for adjusting the coloring.

Supplemental-respectively, carrier material that is utilized may be for example, sodium alginate as a gel promoter for producing a suitable basis, or cellulose derivatives, such as for example, guar-or xanthan rubber, inorganic gel forming agents, as for example aluminum hydroxide or bentonite (so-called thixotrope gel promoters), polyacrylic acid derivatives, such for example, Carbopol®, poly vinylpyrrolidon, microcristalline cellulose or carboxymethyl cellulose. In addition, also amphiphile and low and high molecular compounds such as phospholipids are to be considered. The gels can either be hydrogels on a water basis or can be hydrophobic organogels, for example those on the basis of mixtures of low and high molecular paraffin hydrocarbons and Vaseline. The hydrophilic organogels can be prepared for example on the basis of high molecular polyethylene glycol. These gel-type forms are washable. Among the organogels, the hydrophobic organogels are preferred. Especially preferred are the hydrophobic supplementary agents and additives such as petroleum, wax oleylalcohol, propylenglycolmonostearate and propylene glycolmonopalmitostearate.

If substances in liposomal form are used in the pharmaceutical composition special regulations must be observed with commercially obtained liposomes, in particular, the instructions of the manufacturer must be observed. Liposomes can be water re-suspensions of the lipid vesicles in physiological salt solution, which contain 1% phenoxyethanol as an antimicrobial portion. Any condition which destroys the physical and chemical structure of the liposomes or the material enclosed therein or changing the material enclosed therein is to be avoided. When using such liposomes, a hydrogel can be used as a basis lotion. Hydrophobic materials, in particular those that are oil based, such as ethylene or polyethylene glycol is not a suitable for this purpose.

When processing substances in liposomal form, the following conditions should preferably be avoided. Use of organic solvents or alcohols, surface active components or detergents above their critical micelle concentration; temperatures of about above 25° C., a pH value of about<6.0 and about>8.0; and osmotic pressure through ion concentration, which significantly deviates from a physiological sodium chloride solution (0.9% by weight or 0.15 M Sodium chloride solution.) and foam generating processing steps. All additional components of the formulation are preferably first combined together and in the last step, the liposomes are added. In a preferred embodiment, about 1 to 30 parts by weight, preferably about 2 to 25 parts by weight, especially preferred about 2 to 15 parts by weight of liposomes can be added, in particular, liposomes which contain at least one DNA repair enzyme, with respect to 100 parts by weight of the total composition.

Light protection substances (UV-filter) include light absorbing or light reflecting substances of organic or inorganic structure. As light reflecting substances, for example can be used titanium oxide, titanium dioxide and/or zinc oxide, in particular titanium dioxide. As light absorbing agents, which are also called light filters, UV-B filters, UV-A filters, also broad band filters can be used, which absorb UV-A as well as UV-B radiation. Also, as UV-B filter can used for example, p-amino benzoate acid, cinnamylic acid and benzimidazol derivative, as broad band filters benzophenone-derivatives can be used. As UV-A filter substances that have a dibenzoyl methane structure can be used.

In a preferred embodiment, the light protection substances (UV filter) may be preferably all of those that currently are, or will be certified in the future in countries of the European Union and/or of the United States of America such as for example, octylmethoxycinnamate, zinc oxide, titanium oxide, octocrylen, 4-methylbenzylidencampher, butylmethoxydibenzoylmethane, octylsalicylate, titanium oxide, phenylbenzimidizolsufonate, diethylhexylbutamidotriazone, doctylbutamidotriazone, terephthalyidendicamphersulfonic acid, octyltriazone, ethylhexyltriazone, drometrizole, trisiloxane, benzophenone-3, homosalate and/or isoamylmethoxycinnamate.

Agents for repelling insects and flies, in particular mosquitoes, can be synthetic or natural. Preferred are commercially available agents known to those skilled in the arts. Such agents are among others, referenced in Wilfried Umbach; Cosmetic: Development, Production and Application of Cosmetic Agents, Thieme Press, 1988 ISBN 3-13 712601 0, pp. 135 to 140.

Skin tanning agents as a subgroup of the supplemental agents are understood herein to be substances which effect the artificial tanning of the skin through coloring or through chemical reaction of the horny layer of the epidermis of the skin (defined as follows). They can be natural substances, as for example, extracts from green walnuts skins and/or henna, each of which contain as active substance naphthochinone, in particular, 2-hydroxy-1,4-naphtochinone and/or 5-hydroxy-1,4 naphthochinone. Furthermore, chemical compounds such as hydroketones, hydroxyaldehydes and dicarbonyl compounds, in particular dihydroxyceton can be used.

As tanning accelerators, vitamins, active substances and/or active substance complexes such as for example moisturizer and/or anti-aging complexes can be used, all of which are commercially available and which are known to those skilled in the arts.

In a particular embodiment, the pharmaceutical composition contains about 1 to 25 parts by weight, preferably about 2 to 20 parts by weight, especially about 2 to 15 parts by weight of a component (i), about 1 to 30 parts by weight, preferably about 2 to 25 parts by weight, in particular especially about 2 to 15 parts by weight of component (ii), and as component (iii), about 20 to 40 parts by weight, preferably about 25 to 35 parts by weight glycerin and as a residual component (v) one or more supplements with respect to 100 parts by weight of a composition containing the components (i), (ii), (iii) and (v).

The pharmaceutical composition according to the present invention is for topical application. Application to the skin is the usual form including lotions, emulsions, gels, salves, creams, solutions for applying or spraying of a mixed phase respectively amphiphile emulsions systems (oil water-water/oil mixing phase) as well as liposomes and transfersomes, preferably lotions, emulsions, gels, creams and other solutions for spreading and/or spraying is specially preferred lotions, gels and creams. Preferably, the active substance is applied locally in the area in which the skin change has occurred and/or the disease is present and/or is to be prevented or treated as a prophylactic measure.

Further topical applicable forms are pastes, powders or solutions. The pastes contain oftentimes as a basis for consistency hydrophobic and hydrophilic supplements, however, preferably hydrophobic supplements with a relatively high portion of solids. The topically applicable powders, in order to raise the dispersion properties as well as their flow and gliding capacity and to avoid formation of agglomerates, can contain starches such as wheat or rice starch, flame dispersing silicon dioxide or silica which serve also as thinning agents.

Each of these suitable formulations can then be made according to recipes and methods known to those skilled in the art and on the basis of pharmaceutical and physical knowledge.

A “formulation supplement” as defined herein are all pharmaceutically suitable supplements and carrier agents known to those skilled in the art and which are utilized in the conventional way according to conventional pharmaceutical technical methods and taking into account the different indications and kinds of applications, suitable forms of medicines, pharmaceutical compositions and/or cosmetics.

A further aspect of the present invention refers to a method for producing a pharmaceutical composition comprising (i) a medical drug of nopal cactus and (ii) at least one active substance having light-dermatosis-therapeutic properties comprising the following steps:

providing a medical drug from nopal cactus,

providing at least one active substance having light-dermatosistherapeutic properties,

mixing the medical drug from step a) with the active ingredient from step b) to obtain a pharmaceutical composition having light-dermatosis-therapeutic properties.

A special embodiment refers to the mixing described in c) for producing a pharmaceutical composition, with mixing about 1 to 25 parts by weight of b) of medical drug from step a) with about 1 to 30 parts by weight of at least one DNA-repair enzyme containing liposomes from step b) with respect to 100 parts by weight of the total composition and at least one further suitable supplement as an a residual portion.

The pharmaceutical composition according to the invention can be used for treatment of light-dermatosis, wherein the light-dermatosis may be an acute and/or chronic light-dermatosis, in particular, a reddening of the skin, an inflammation of the skin and/or a DNA damage and/or as the result therefrom, especially dermatitis solaris, tissue degeneration, benign and/or a malignant cell changes.

Furthermore, the pharmaceutical composition can be used as a cream, sun lotion, sun gel, after-sun cream, after-sun lotion, after-sun gel and/or as a spray solution for treatment and/or prevention of light-dermatosis. Galenic formulations of such composition are known to those skilled in the art and are also listed, among others, in Wilfried Umbach; Cosmetic: Development, Production, and Application of Cosmetic Agents, Thieme Verlag 1988, ISBN 3-13 712601 0 page 118-130 as well as Rudolf Voigt, Pharmaceutical Technology, 9th Edition, German Pharmacists Press, Stuttgart 2000, in particular Chapters 17 and 18.

In a further aspect, the pharmaceutical composition of the present invention may also be added as an active substance complex for medical, pharmaceutical and/or cosmetic products for treatment and/or prevention of light dermatoses. Accordingly, medical products can be in the form of such medical products as are subject to regulations and pharmaceutical products in the form of pharmaceutical products subject to regulations for drugs and medication and/or can also be pharmaceutical compositions as commercial over-the-counter pharmaceutical compositions.

The term “cosmetic products”, as used herein encompasses, for example, the conventional salves, creams, lotions, gels, solutions, sprays, lip sticks for cosmetic, in particular, for beauty care and/or decorative cosmetic application.

In a preferred embodiment, the pharmaceutical composition according to the invention can be applied as an active substance complex in an amount of about 0.1 to 50 parts by weight, preferably about 1 to 30 parts by weight, especially preferred about 1 to 10 parts by weight with respect to 100 parts by weight of the medical, pharmaceutical and/or the cosmetic products for treatment and/or prevention of light-dermatosis.

In yet a further aspect, the pharmaceutical composition according to the invention can be utilized to enhance the skin tanning and/or for enhancement of pigment formation in the skin.

“Skin tanning” as herein used is tanning of the skin with and without UV radiation. UV-B radiation results in pigmentation of the skin as well as “indirect pigmentation” caused by UV-B radiation as well as direct pigmentation as a result of UV-A radiation.

Tanning of the skin without UV radiation can be divided into three groups, the decorative coloring of the skinned with removable make-up preparations, coloring of the skin through regular intake of carotene preparations as well as artificial skin tanning through coloring or through chemical change in the horny layer of the epidermis with the so-called self tanning agents (see also Wilfried Umbach, Cosmetic: Development, Production and Application of Cosmetic Agents, Thieme Press, 1988 ISBN 3-13 712601 0, pp. 130 to 133)

The medical drug from nopal, especially in combination with at least one DNA repair enzyme, results in skin tanning through enhancement of the normal, regular pigment formation as a result of UV radiation (see FIG. 1).

The afore-described embodiments can be used alone or in combination with other embodiments.

The following figures and examples are presented to explain the invention further without limitation. The patent claims that follow are incorporated herein by reference into the entire description.

### BRIEF DESCRIPTION OF THE DRAWING

Other features and advantages of the present invention will be more readily apparent upon reading the following description of currently preferred exemplified embodiments of the invention with reference to the accompanying drawing, in which:

FIG. 1 shows the effect of a pharmaceutical composition formulated as an after-sun lotion to a sunburn reaction.

FIG. 2 shows the effect of the pharmaceutical composition formulated as an after-sun lotion to the tanned skin.

### DETAILED DESCRIPTION OF PREFERRED EMBODIMENTS

Throughout all the Figures, same or corresponding elements are generally indicated by same reference numerals. These depicted embodiments are to be understood as illustrative of the invention and not as limiting in any way.

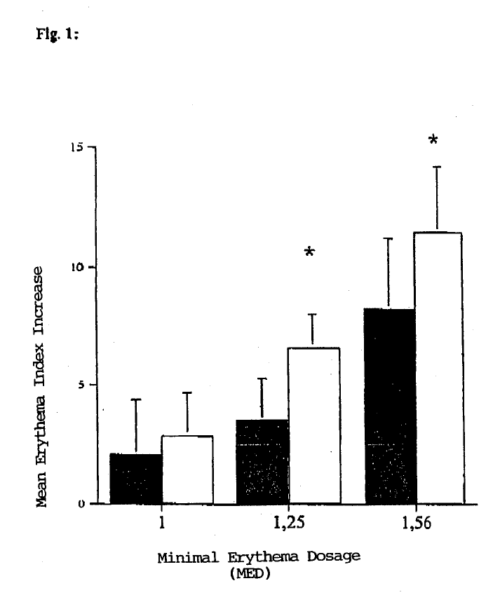

Turning now to the drawing, and in particular to FIG. 1, there is shown a graph showing an accelerated healing of the reddened skin after topical application of the composition, as compared to the untreated skin 24 hours after artificial sun-simulated UV irradiation to the skin in the laboratory on volunteer participants with 1.0; 1.25 and 1.56 minimal erythema dosages (minimal reddening of the skin- respectively, sunburn dosages). The X-axis shows the minimal erythema dosage (MED) and the Y-axis, the mean erythema index increase (24 h) (see methods part: reflexspectroscopy). The reddening of the skin was measured by means of reflexspectroscopy on a total of 7 participants (4 women, 3 men, average age 44 years, range between 33 to 59 years) after irradiating 1 cm in diameter skin areas at their buttocks. *p<=0.001; pharmaceutical composition formulated as an after-sun lotion, versus untreated skin (student-T test). The white bars herein show the erythema index increase of the untreated skin (control) and the dotted, dark bars, show the erythema index increase in the skin treated with the pharmaceutical composition formulated as an after-sun lotion of the participants after UV irradiation.

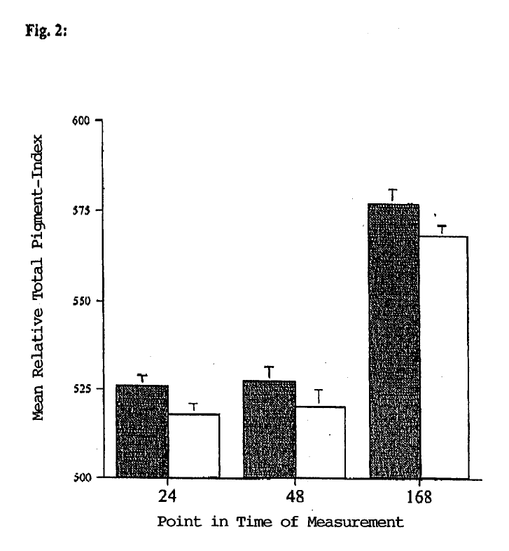

The graph in FIG. 2 shows an increased skin tanning through topical application of the composition as compared to untreated skin with the same participants as in FIG. 1. The X-axis shows the point in time of measurement (at 24, 48 and 168 hours after UV radiation). The Y-axis shows the mean relative total pigment-index (see methods: reflexspectroscopy). A relative value for the skin pigmentation was determined by means of reflexspectroscopy 24 and 48 hours as well as seven-day (168 hours) after UV radiation. The white bars show the relative total pigment-index of untreated skin (control) and the dotted, dark bars, show a relative total pigment-index after UV irradiation of skin of the participants treated with the pharmaceutical composition and formulated as an after-sun lotion

### Example 1

A study of the pharmaceutical composition formulated as after-sun lotion.

The effect of the pharmaceutical composition formulated as after-sun lotion was tested with 7 volunteer participants (4 women, 3 men: average age 44 years, range 33 to 59 years). In this study, a pharmaceutical composition with the following components was tested:

### Components of the After-sun Lotion

The after-sun lotion contains herein 5 parts by weight of the preferred active substance complex as set forth in Example 2 with respect to 100 parts by weight of the total composition of the after-sun lotion.

| | Data expressed in parts per |
|---|---|
| | weight per 100 parts per weight |
| Components | of the total composition |
| Nopal | 0.3 |
| Micrococcus-Lysate in liposomal for | 0.25 |
| containing UV-Endonuclease | |
| (Ultrasome) | |
| Plankton extract consisting of Anacyst | 0.25 |
| nidulans in liposomal form, containi | |
| photolyase (Photosome) | |
| Panthenol | 0.30 |
| Tocopherolacetate | 0.30 |
| Lecithin | 0.01 |
| Soy Glycin | 0.50 |
| Methylparaben | 0.18 |
| Allantoin | 0.10 |
| Butylparaben | 0.06 |
| Ethylparaben | 0.03 |
| Propylparaben | 0.03 |
| Capryl Acid/Caprin Triglyceride | 5.00 |
| Glycerin | 5.0 |
| Decylglucoside | 1.10 |
| Buxus Chinensis | 1.00 |
| Natriumcarbomer | 0.75 |
| Squalan | 0.50 |
| Persea Gratissima | 0.50 |
| Trilaureth-4-phosphate | 1.00 |
| Water | filled up to 100 parts per weight |

### UV-lrradiation

The UV irradiation was carried out by means of an Oriel 1000-watt sun simulator (Oriel Corp., Darmstadt, Germany) provided with a dichroism mirror with a glass filter of WG320 1 mm and UG 5/1 mm glass filter. The intensity of the light source was monitored, respectively measured through regular measurements during this study with a broadband-thermopile radiometer (Dexter Research 2M model with quartz window) (Medical Physics Dryburn Hospital, Durham U.K.). This radiometer was calibrated prior thereto by the regional medical physics department, Royal Victoria infirmary unit (Newcastle upon Tyne) under utilization of a reference Thermopile radiometer (Hilgar-Swartz FT 17). The UV radiation intensity measured during the entire study was 12.0 mW/cm² as measured at a distance of 20 cm from the outer convex lens of the system. This UV intensity was kept constant during this study with a built-in automated photo feedback system of the Oriel sun simulator. The wavelengths spectrum of the Oriel sun simulators were measured with an International Light Spectroradiometer (International Light Inc., Newburyport, Mass., U.S.) and corresponds to die regulation of the American health administration (Food and Drug Administration (FDA)) and the European COLIPA (Comite de Liaison des Assocations Europeenne de L'lndustrie de la Parfumerie, des Produits Cosmetiques et de Toilette)-norm for the determination of light protection factors of light protection preparations.

### Minimal Erythema Dosage (MED) Testing

Determination of MED was measured on the participants through the UV irradiation of a total of 6 skin areas each measuring about 2 cm at the untanned buttocks in accordance with regulations of the American FDA and the European COLIPA-norm. The MED testing was carried out without light protection preparations, wherein immediately after UV radiation, the lower part of the radiated area was treated with the pharmaceutical composition according to the invention formulated as an after-sun lotion where the concentration of the application was 2 mg/cm².

### Reflexspectroscopy

Skin reddening- and pigmentation where quantitatively determined in the area of the irradiated skin areas by means of reflexspectroscopy utilizing a DermaSpectrometer® (Cortex Technology, Hadsund, Denmark). This apparatus measures a relative value, respectively index for the reddening (erythema) and pigmentation of the skin based on the absorption characteristics of the skin. The erythema-index increase as shown in FIG. 1 was determined through subtraction of the erythema value of the non-irradiated skin from the erythema-index value of the irradiated skin areas of the same participants. The total pigment-index was computed through summation of the measured values at the set time points for each of the participants.

### Results

The results of the study are represented in FIG. 1 and 2. The administration of the pharmaceutical composition formulated as after-sun lotion and containing a medical drug from nopal as well as ultrasomes and photosomes as DNA-repair enzymes led to a significant reduction of the UV induced sunburn reaction (FIG. 1), whereby at the same time, tanning of the skin was not prevented but on the contrary, tanning was even slightly increased (FIG. 2).

The pharmaceutical composition formulated as after-sun lotion with medical drug from nonal in combination with ultrazomes and photozomes accelerated the healing of the acute sunburn reaction (FIG. 1). Remarkably, this anti-sunburn effect does not coincide with the reduction of skin tanning, as would be expected, for example when applying regular light protection preparations. In contrast thereto, the application even leads to a slight increase of tanning (FIG. 2). This increase in tanning due to UV irradiation is not based on a coloring of the skin but most probably is due to the pharmaceutical composition formulated as after-sun lotion with nopal in combination with ultrasomes and photosomes fortified products and/or release of melanin. (skin pigment). This conclusion is based among others, that the treatment of the skin by means of the after-sun lotion of the skin with the medical drug nopal in combination with ultrasomes and photosomes without UV irradiation does not lead to a coloring of the skin.

### Example 2

Active substance complex, which is suitable as a supplement for medical, pharmaceutical and/or cosmetic products,

The preferred active ingredient contains:

| Component | Parts per Weight |
|---|---|
| Glycerin | 30.0 |
| Nopal powder | 6.0 |
| Ultrasome | 10.0 |
| Photosome | 10.0 |
| Potassiumsorbate | 0.3 |
| Sodiumbenzoate | 0.2 |
| Water | 43.6 |

For the production of a preferred active ingredient complex, Nopal powder is presented in glycerin and the respective amount of water stirred into.

Subsequently, the ultrasomes and the photosomes are added under stirring until a homogenized mass has formed. Furthermore, potassium sorbate and sodium benzoate are likewise added separately under stirring of the mixture and stirred until a homogenous mass has formed.

While the invention has been illustrated and described in connection with currently preferred embodiments shown and described in detail, it is not intended to be limited to the details shown since various modifications and structural changes may be made without departing in any way from the spirit of the present invention. The embodiments were chosen and described in order to best explain the principles of the invention and practical application to thereby enable a person skilled in the art to best utilize the invention and various embodiments with various modifications as are suited to the particular use contemplated.

What is claimed as new and desired to be protected by Letters Patent is set forth in the appended claims and their equivalents:

What is claimed is:

1. A pharmaceutical composition for the prevention and treatment of a light-dermatosis comprising (i) a medical drug form nopal cactus, and (ii) at least one active substance with light-dermatosis therapeutic properties.

2. The pharmaceutical composition of claim 1, wherein the light-dermatosis is at least one of an acute and a chronic light-dermatosis.

3. The pharmaceutical composition of claim 2, wherein the acute light-dermatosis to be treated is from the group consisting at least one of skin reddening, skin inflammation and DNA damage.

4. The pharmaceutical composition of claim 2, wherein the acute light-dermatosis to be treated is at least one of dermatitis solaris (sunburn) and sun allergy.

5. The pharmaceutical composition of claim 2, wherein the chronic light-dermatosis is associated with at least one selected from the group consisting of DNA-damage, tissue degeneration, premature skin aging, benign or malignant cell changes.

6. The pharmaceutical composition of claim 1, wherein the light-dermatosis therapeutic properties are selected from the group consisting of inflammation inhibiting-, skin soothing- and DNA-damage repairing-properties.

7. The pharmaceutical composition of claim 1, wherein the active substance is at least one of anti-inflammatory and skin soothing.

8. The pharmaceutical composition of claim 7, wherein the active substance is selected from the group consisting of bisabolol, panthenol, dexpanthenol, reishi (ganoderma lucidum), allantoin, vitamin E, green tea-extract, chamomile, aloe vera and witch hazel.

8. The pharmaceutical composition of claim 1, wherein the active substance is one having DNA-damage repairing properties and is at least a DNA-repair enzyme.

9. The pharmaceutical composition of claim 8, wherein the at least one DNA-repair enzyme is selected from the group consisting of endonuclease V, O⁶-methylguanine-DNA-methyl transferase, photolyase, uracil-and hypoxanthien-DNA-glycosylase, apyrimidin/apurin-endonuclease, DNA-exonuclease, damaged-baseglycosylase, 3-methyladenine-DNA-glycosylase and correndonuclease;

10. The pharmaceutical composition of claim 8, wherein the at least one DNA-repair enzyme is contained at least in one of micrococcus luteus and anacystis nidulans extract.

11. The pharmaceutical composition of claim 8, wherein the at least one DNA-repair enzyme is contained in a carrier material selected from the group consisting of liposomes, nanocapsules, nanoparticles, and microparticles.

12. The pharmaceutical composition of claim 9, wherein the at least one DNA-repair enzyme is endonuclease V, photolyase or is contained in liposomal form in at least one of micrococcus luteus-and in anacystis nidulans extract.

13. The pharmaceutical composition of claim 12, wherein the medical drug is in combination with at least one of endonuclease V, photolyase and contained at least one of liposomal form in micrococcus luteus-and anacystis nidulans extract.

14. The pharmaceutical composition of claims 1, wherein the composition contains about 1-40 parts by weight of the compound (i) and about 1-40 parts by weight of the compound (ii), and as residual portion (v) one or more additives, with respect to 100 parts by weight of a composition having compounds (i), (ii) and (v).

15. The pharmaceutical composition of claims 14, wherein the composition contains about 2-25 parts by weight of compound (i) and about 2-25 parts by weight of compound (ii).

16. The pharmaceutical composition of claims 14, wherein the composition contains about 2-15 parts by weight of compound (i) and about 2-15 parts by weight of compound (ii).

17. The pharmaceutical composition of claim 1, wherein the composition contains about 1-40, of the compound (i), about 1-30 parts by weight, of the compound (ii), and further comprising about 20-40 parts by weight of a compound (iii), and as residual portion (v) one or more additives, with respect to 100 parts by weight of a composition having compounds (i), (ii), (iii) and (v).

18. The pharmaceutical composition of claim 17, wherein the composition contains about 2-25 parts by weight of compound (i), about 2-25 parts by weight of compound (ii) and contains about 25-35 parts by weight glycerin as compound (iii).

19. The pharmaceutical composition of claim 18, wherein the composition contains about 2-15 parts of compound (i), and about 2-15 parts by weight of compound (iii)

20. The pharmaceutical composition of claim 1, wherein the medical drug from nopal-cactus is one derived from at least one of cactus branches and fruits.

21. The pharmaceutical composition of claim 20, wherein the medical drug from nopal cactus is derived from comminution of spikeless cactus branches.

22. A method for producing a pharmaceutical composition for treatment of light-dermatosis comprising (i) a medical drug from nopal cactus and (ii) at least one active substance having light-dermatosis therapeutic properties, comprising the following steps:
a) providing a medical drug from nopal-cactus,
b) providing at least one active substance with light-dermatosis therapeutic properties from the group consisting of bisabolol, panthenol, dexpanthenol, reishi (ganoderma lucidum), allantoin, vitamin E, green tea-extract, chamomile, aloe vera, witch hazel and a DNA-repair enzyme, and
c) mixing the medical drug from step a) with the active substance of step b) for obtaining a pharmaceutical composition having light-dermatosis-therapeutic properties.

23. The method of claim 22, wherein the medical drug from nopal-cactus is obtained from at least one of the cactus branches and fruits, in particular the cactus branches.

24. The method of claim 23, wherein the medical drug from nopal-cactus is obtained from comminution of spikeless cactus branches.

25. The method according to claim 22, wherein the mixture comprises approximately 1-25 parts by weight of the medical drug from step a) and approximately 1-30 parts by weight of at least one liposome containing DNA-repair enzyme of step b) each with respect to 100 parts by weight of the total composition, and at least one suitable additive as residual compound.

26. The method of claim 25, wherein the DNA-repair enzyme is at least one of endonuclease V and a photolyase.

27. The method of claim 26, wherein the DNA repair enzyme is contained in micrococcus luteus extract and/or anacystis nidulans extract.

28. A method for treatment or prevention of a light dermatosis comprising the steps of: applying a pharmaceutical composition with light-dermatosis properties to an area of the skin affected by light dermatosis, wherein the pharmaceutical composition contains a medical drug from nopal cactus and at least one active substance with light-dermatosis therapeutic properties.

29. The method of claim 28, wherein the light-dermatosis is at least one of an acute light-dermatosis and a chronic light-dermatosis.

30. The method of claim 28, wherein the light dermatosis is an acute light-dermatosis exhibiting symptoms selected from the group of skin reddening, skin inflammation, DNA-damage, dermatitis solaris (sun burn) and sun allergy.

31. The method of claim 28, wherein the DNA-damage exhibits symptoms of the type selected from the group of tissue degeneration, premature skin aging, benign and malignant cell changes.

32. The method of claim 28, wherein the pharmaceutical composition is contained in a carrier selected from the group consisting of sun cream, sun lotion, sun gel, after-sun cream, after-sun lotion, and after-sun gel.

33. The method of claim 28, wherein an active complex for medical, pharmaceutical and/or cosmetic products for treatment and/or prevention of a light-dermatosis and at least one active substance with light-dermatosis therapeutic properties.

34. The method of claim 33, further comprising a carrier suitable for topical application to the skin.

35. The method of claim 28, wherein the pharmaceutical composition comprises an active substance complex in an amount of approximately 0.1-50 parts by weight, preferably approximately 1-30 parts by weight, in particular approximately 1-10 parts by weight with respect to 100 parts by weight of the medical, pharmaceutical and/or the cosmetic products for treatment and/or prevention of a light-dermatosis.

36. The method of claim 28, wherein the pharmaceutical composition is utilized for enhancement of skin tanning.

37. The method of claim 28, wherein the pharmaceutical composition is utilized for enhancement of pigment formation in the skin.
